(19) United States
(12) Patent Application Publication
(10) Pub. No.: US 2014/0350221 A1
(43) Pub. Date: Nov. 27, 2014

(54) METHOD FOR DETERMINING INTRINSIC BINDING PARAMETERS OF AN ANALYTE TO A LIGAND, A METHOD FOR SELECTING AN ANALYTE FROM A GROUP OF ANALYTES, THE SELECTED LIGAND OR ANALYTE, AND SENSOR

(71) Applicants: Richard Bernardus Maria Schasfoort, Haaksbergen (NL); Gerard Henricus Maria Engbers, Oldenzaal (NL)

(72) Inventors: Richard Bernardus Maria Schasfoort, Haaksbergen (NL); Gerard Henricus Maria Engbers, Oldenzaal (NL)

(21) Appl. No.: 14/116,813

(22) PCT Filed: May 11, 2012
(86) PCT No.: PCT/EP2012/058840

§ 371 Date: Feb. 24, 2014

(30) Foreign Application Priority Data
May 11, 2011 (EP) 11165769.8

(51) Int. Cl. G01N 33/543

(52) U.S. Cl. CPC G01N 33/543; G01N 2500/20
USPC 530/350; 436/501; 422/69; 506/9

(57) ABSTRACT

The present invention relates to a method for determining intrinsic binding parameters, such as K_{D}, k_{d} and kₐ, of an analyte to a ligand, such as a drug and a protein, a drug and a receptor, and an antibody and antigen, wherein the maximal binding response Rₘₐₓ or R_{L} and at least one binding parameter is determined at at least two different ligand surface densities present on a sensor support, and extrapolating the value of the binding parameter to ligand density=0, characterized by Rmax=0 or R₁=0, to a method of selecting an analyte and/or ligand, and to the selected ligand, analyte and a sensor.

The present invention relates to a method for determining the intrinsic binding parameter of an analyte to a ligand, to a method for selecting an analyte from a group of analytes, to the analyte or ligand selected, and to a sensor for these methods.

In the development of drugs and in relation to their use for a therapy and prophylaxis it is important to know the intrinsic binding parameters of an analyte to a ligand. In this respect the analyte and the ligand may comprise a drug and a protein, a drug and a receptor and an antibody and an antigen. Evidently, dependent on the intended use the ligand and analyte may be interchanged, such as the binding of antibody as ligand to an antigen as analyte. Similarly, a ligand may be bound to the sensor support and the ligand present a solution to be contacted with the immobilized analyte. But by definition, the molecule immobilized to a support which is in this case a sensor support is called the ligand, and the molecule in solution is called the analyte and is to be contacted with the ligand in order to bind and thereby determine the binding parameters, such as the affinity and dissociation constants, the equilibrium dissociation constant (K_{D}), the equilibrium association constant (K_{A}=1/K_{D}), the dissociation rate constant (k_{d}) and the association rate constant (kₐ).

The binding parameters, such as the affinity constant of such interaction pair, may be determined by various conventional techniques, such as isothermal calorimetry, fluorescence anisotropy, and the like. Presently preferred are determination techniques which are used without a labeling of either the ligand or the analyte. The biological interaction in between the ligand and the analyte is then measured at a sensor surface of the sensor support, such as by Surface Plasmon Resonance.

The binding of the analyte to the ligand results in a measurable change in response of the biosensor which is recorded as function of time. This recording results in a so called sensorgram.

The transport of the analyte to the sensor surface for binding to the immobilized ligand comprises the kinetics of binding to the ligand and releasing the analyte form the ligand. This kinetic binding and releasing is dependent on the flow rate of the analyte over the sensor surface, and in particular dependent on the ligand surface density. In addition, it has to be born in mind that biological molecules, such as immunoglobulines (Igs) comprise two or more binding sides for the analyte, such that there is no 1:1 interaction in between on the one hand the ligand and on the other the analyte.

The binding parameters, such as the affinity constant for a particular ligand is acceptably determined by a given surface density at which the ligand is then exposed to two or more analyte concentrations. The resulted sensorgrams are subjected to a fitting procedure resulting in a determination of the binding parameters, and also of the Rₘₐₓ which represents to binding of analyte to all available ligands on the sensor surface.

This method of determining binding parameters has several drawbacks. One drawback is rebinding. When an analyte binds to the ligand a certain Rₘₐₓ is found. The analyte may be released subsequently and thereafter may rebind to another ligand molecule. However, in this process of binding releasing and rebinding, the analyte does not leave the evanescent field or window for measurement by the biosensor. Accordingly, the release of the analyte cannot be measured. This means that the dissociation rate will be measured as too slow, which may result in a difference with the actual intrinsic dissociation equilibrium constant (and dissociation rate).

Another drawback is that the 1:1 interaction (as indicated above) does not always exist. If the analyte or ligand comprises two binding sides (of equal or different binding strengths) a so called biphasic performance is the result. Further, a binding via the two binding sides will occur and subsequently when more analyte molecules are bound or captured, one of the binding sides (the less binding strength side) will be released and a further analyte molecule bound. This problem is in particular relevant for antibodies and antigens.

Additionally a physical rate determining process called mass transport limitation occurs when analyte molecules diffuse in the so-called stagnant layer which influences the association rate constant of binding of the analyte to the ligands immobilized on the sensor surface. During injection of the analyte under laminar flow conditions the analyte diffuses in this unstirred stagnant layer and will be captured by the surface. When the analyte concentration is relatively low and the capacity of the sensor is high the kinetic process is limited diffusion or limited mass transport. This results in an initial linear binding process (delta Refractive Units (RU)/second) that depends on the concentration of the analyte, temperature and flow conditions determining the thickness of the stagnant layer. When the ligand density is lower the mass transport limitation effect is less.

Accordingly, there may be problems in calculating rate- and affinity constants of biomolecular interactions. It is well known that the apparent rate- and affinity constant k_{d}, kₐ and K_{D} will change significantly if rebinding effects, biphasic behavior, mass transport limitation and/or steric hindrance of the immobilized/captured molecules occur at a sensor chip surface of the sensor support.

The present invention has for its object to avoid these problems, and thereby make it feasible to determine the intrinsic binding parameters of an analyte to a ligand. This means, that the determined binding parameters are no longer dependent on the defects of the currently used method of determination and in particular not dependent on the conditions under which the determination has been carried out (such as density of ligand, concentrations of analyte, and type of interaction in between ligand and analyte).

The method of the invention applies an array of various ligand surface densities and the analyte is exposed to the array resulting in a single rate- and affinity constant by extrapolating the value for the binding parameter to be measured, such as the apparent k_{d} and K_{D} values to Rₘₐₓ=0, or to R_{L}=0. Theoretically this means that the affinity constant of a single ligand molecule to its single analyte molecule is determined in the limit to zero response.

According to the invention is provided a method for determining intrinsic binding parameters, such as K_{D}, k_{d} and kₐ, of an analyte to a ligand, such as a drug and a protein, a drug and a receptor, and an antibody and antigen, wherein the maximal binding response Rₘₐₓ and at least one binding parameter is determined at at least two different ligand surface densities present on a sensor support, and extrapolating the value of the binding parameter to Rₘₐₓ=0 or to R_{L}=0.

The Rₘₐₓ value reflects the analyte binding capacity proportional to the ligand density. If the ligand cannot be immobilized directly or is impure, an anti-ligand ligand is coupled to a sensor support and the ligand is captured by the anti-ligand. Then a two-step interaction process should be measured; first capturing the ligand of interest followed by analyte binding to the captured ligand. In this case the ligand density is measured as a captured value called R_{L} and extrapolation to ligand density approaching to zero can be performed. The R_{L} value can also be determined if the level of immobilization of the ligand is known from the degree of binding of the ligand to the sensor surface. In other words the biomolecular interaction rate and equilibrium parameters can be calculated by either extrapolating to the calculated Rₘₐₓ=0 or the measured R_{L}=0 (in a captured or direct way) or both.

The invention is based on the insight that when measuring the binding of an analyte to a ligand which is present on the measuring surface of a sensor support in decreasing surface densities, problems in relation to rebinding, mass transport limitation and/or biphasic behavior are corrected or circumvented, and determination is far more closely under conditions of an 1:1 interaction in between the ligand and analyte.

Using an acceptable extrapolation method, the ligand density approaching to zero is characterized by Rₘₐₓ=0 or R_{L}=0 value may be determined from the various sensorgrams and results in the intrinsic binding parameters which are characteristic for the particular ligand/analyte combination. It is at this ligand density in the limit to zero condition, R_{L}=0 or Rmax=0, that the analyte binds to one ligand molecule at the surface of the sensor support and substantially avoid other ligands and/or analyte molecules. Accordingly, the present invention provides a method for determining intrinsic binding parameters, such as K_{D}, k_{d} and kₐ, of an analyte to a ligand, such as a drug and a protein, a drug and a receptor, and an antibody and antigen, wherein the maximal binding response Rₘₐₓ and at least one binding parameter is determined at at least two different ligand concentrations, and extrapolate the value of the binding parameter to Rₘₐₓ=0 or R_{L}=0.

It is noted that by definition the ligand is bound to the measuring surface of the sensor support whereas the analyte is provided at different concentrations for binding to the immobilized ligand. Accordingly, the ligand may be an antibody or an antigen and in turn the analyte is then the antigen and the antibody, respectively. The same holds for ligands being a drug, a protein or a receptor. R_{L} is the response of ligand capturing, and corresponds to the number of analyte binding sites per spot. The data points are fitted with the exponential equation y=a e^{bx}. By extrapolation of R_{L}=x to 0, rate constants k_{d}^{R0}, kₐ^{R0} and an dissociation equilibrium constant K_{D}^{R0} can be determined approaching to a single analyte molecule interacting with a single ligand molecule. Instead of using the R_{L} value the calculated Rₘₐₓ values from the analyte responses, can be used for extrapolating the k_{d} and K_{D} values to zero response. The Rₘₐₓ value is the theoretical response value when all ligand molecules are saturated with analyte molecules. The ratio of the calculated Rₘₐₓ and R_{L} is constant and is proportional to the molecular weights: Mw_{analyte}/Mw_{Ligand}=Rₘₐₓ/R_{L}. A cross-check can be performed by comparing the ratio of the calculated Rₘₐₓ and the experimentally determined R_{L} for extrapolation R_{L} to zero or Rₘₐₓ to zero. Theoretically the values for extrapolation R_{L} to zero or Rₘₐₓ to zero should be equal.

Obviously, the method for determining the intrinsic binding parameters is more reliable when the analyte is subjected to more than two different ligand concentrations or surface densities. Preferably, are used 3-10, and more practically 3-6 different ligand concentrations or surface densities.

When determining the binding parameter by two different ligand surface densities or serial surface dilutions of the ligand, the binding parameters at R_{L}=0 or Rₘₐₓ=0 may be determined by extrapolation. However, it has been found that exponential fit is advantageously preferred, because with exponential fit the influence of lower R_{L} or Rₘₐₓ values is better taken into account.

As indicated hereinbefore, it is preferred that the binding parameter is determined using a 1:1 interaction model. This model is not only relatively straight forward, but the determination of binding parameters at R_{L} or Rₘₐₓ=0 also is based on the 1:1 interaction between ligand and analyte.

In many cases if the ligand cannot be immobilized directly or is impure, an anti-ligand ligand is coupled to a sensor support and the ligand is captured by the anti-ligand. The signal R_{L} can be used for the extrapolation.

Examples of anti-ligands include but are not limited to streptavidin, neutravidin, avidin, protein A, protein G, anti-IgG, and the like. When the ligand is a so-called tag then the anti-ligand is an anti-tags, for instance anti-tags against tags including but not limited to his-tag, FLAG-tag, CBP-tag, MBP-tag, GST-tag, HA-tag, myc-tag, isopep-tag, BCCP, calmodulin-tag, nus-tag, green fluorescent protein-tag, thioredoxin-tag, S-tag, Softag, SBP-tag, Ty-tag, DNA-tag and the like.

For determination it may be advantageous to use a mixture of labeled and non-labeled analytes, such as biotinylated and non-biotinylated. The different types of analytes may be used sequentially or as a mixture in the binding experiments.

If the determination is to be carried out in an aqueous medium and/or the sensor support has non-optimal properties then it may be beneficial to attach a intermediate layer, such as a hydrophilic layer, to the sensor support and bind the ligand to the sensor support directly or indirectly via the intermediate (hydrophilic) layer such as hydrogel matrix or planair monolayer or thin layer. It may be preferred to first bind an anti-ligand to the sensor support or to the intermediate layer, such as the hydrogel matrix, covalently which anti-ligand may then capture the ligand before the determination has been carried out. In order to capture a ligand density series the anti-ligand can be immobilized in an anti-ligand density serial dilution. In another way the anti-ligand can be coupled as an homogeneous capture surface and the ligand can be captured in a gradient density series. Such affinity capturing, by for instance streptavidine or via tags, is a well accepted method for binding a ligand to a solid support.

According to a preferred embodiment is the ligand distributed in a surface layer with such thickness that results in R_{L} values of 5-3000 RU (Refractive Units) for antibodies preferably in the range of 50-500 RU. For antibodies, with a molecular weight of ˜150 kD, this corresponds to ligand surface densities of 5 pg/mm² to 3 ng/mm² and 50-500 pg/mm² respectively. Thereby is formed a 3-dimensional layer in which the ligand is homogeneously or inhomogeneously distributed. This will yield to a larger binding capacity per surface area

According to a preferred embodiment an analyte concentration is exposed to the different ligand surface densities or concentrations at the same time, and generating sensorgrams for each ligand surface density or concentration. Accordingly, it is possible to carry out the determination at higher speed with less injections of various analyte concentrations and in a more reliable manner.

The different ligand surface densities may be formed by distinct surface areas each having a different ligand surface density. However, it is also possible to provide the sensor support with two or more surface areas each comprising extending is one or both directions of the surface area a gradual and/or stepwise change in the ligand surface density. Obviously, the same result will also be obtained with ligands bound indirectly via an anti-ligand or anti-tag to the sensor support.

If, at least two different types of ligands are used and at least two different analytes are used in a mixture or sequentially used it is possible to carry out more ligand to analyte interactions at the same time. In such cases it is preferred that the extrapolation is done by local or global fitting, preferably by local fitting. Local fitting means that from each sensorgram the rate and equilibrium values are determined while global fitting means that the sensorgrams from several analyte injections are grouped and a so called “global” value of the kinetic rate and affinity constant is determined. In other words when global fitting is carried out using various analyte concentrations, then per ligand surface density or concentration only one value for Rₘₐₓ and for binding parameters can be calculated. Accordingly, it is advantageous to use local fitting because also outcasts can be easily even automatically be rejected. Furthermore, provides local fitting a rather large collection of measuring points which may result in a more reliable and easier determination of Rₘₐₓ=0 by extrapolation.

In relation to global fitting it is noted, that the contribution of the sensorgrams from the higher analyte concentrations contribute equal to the “global” value while using local fitting parameters in the extrapolation process the lower analyte concentration (approaching better the single molecular binding of an analyte to a ligand) contribute more to the “true” affinity constants.

When the biosensor not only comprises two different ligand surface densities or concentrations, but the determination is also carried out at at least two analyte concentrations, a very elegant determination method of the intrinsic binding parameters is provided.

When it is required to determine more than one intrinsic binding parameter, such as the equilibrium dissociation constant and/or the dissociation and association rate, it may be preferred that at least two binding parameters of each sensorgram are determined at the same time followed by extrapolation the value of binding parameter to R_{L} or Rₘₐₓ=0.

As indicated before, any label-free interaction analysis technique may be used for determining the intrinsic binding parameters according to the present invention. Such techniques comprise Quartz Cristal Microbalance (QCM) Surface Acoustic Wave, interferometry such as Young and Mach Zehnder interferometers, bilayer interferometers but preferably Surface Plasmon Resonance (SPR).

Another aspect of the invention relates to a method for selecting an analyte or group of analytes for binding to a ligand, comprising the step of:
i) determining an intrinsic binding parameter of the analyte or group of analytes according to a method as claimed by any of the claims 1-9; and
ii) select the analyte or analytes with the better intrinsic binding parameter.

When having determined the intrinsic binding parameter of various analytes for the same ligand or for various ligands, then it is possible to determine the best or optimal analyte for the ligand. This means, that the ligand or analyte would be the best for inhibit a physiological status, pathological status or illness.

Still another aspect of the invention relates to the analyte (or ligand) which is selected according to this method and is then optimal for the treatment of a physiological status, pathological status or illness.

A final aspect of the invention relates to the sensor suitable for use in the method according to the invention for determining intrinsic binding parameters of an analyte to a ligand. Such sensor is specifically intended for use in Surface Plasmon Resonance (SPR). Surface plasmon resonance is suitable for detecting in real-time and label free biomolecular interactions of the specific analyte to a ligand that is immobilized on a sensor having the form of a microchip. For instance the imaging feature of the IBIS-iSPR instrument enables to detect e.g. hundreds of interactions, such as 120, simultaneously.

The surface areas may have the form of circular, rectangular spots, dots and the like, with a dimension of 10-1000 μm, preferably 50-600 μm, more preferably 100-300 μm.

The ligand may be bound directly or indirectly via an anti-ligand or anti-tag to the sensor support. Preferably, the sensor support comprises substrate surface comprises a gold coating (thickness 10-100 Å, such as 30-70 Å, like 50 Å, and/or titanium or chromium coating (thickness 1-5 Å). This provides the best test results in the SPR measurements. The ligand is directly or via the anti-ligand or anti-tag bound to the substrate surface or via an intermediate layer, such as a hydrophilic layer of a desired thickness.

The surface areas or spots of the desired ligand surface density may be oriented in an array using a spotter, such as an array spotter. When the surface comprises various ligand surface densities in a gradual, stepwise or random order then the surface areas may have a larger size or non-circular or elongated form. For example 5-1000 spots can be arranged in an array by using an array spotter. Preferably a continuous flow microspotter is used that can spot different arrays, for instance an array of 48 spots (6×8 array) or 96 spots (8×12 array).

The sensor support may also comprise reference and blank surface areas or spots. These spots may be used for calibration, for compensation of non-specific binding

Mentioned and other characteristics and features of the method according to the invention will be further illustrated and discussed with reference to the following examples which are given for information purposes only and not intended in any respect to limit the invention. In this respect reference is made to the figures, wherein:

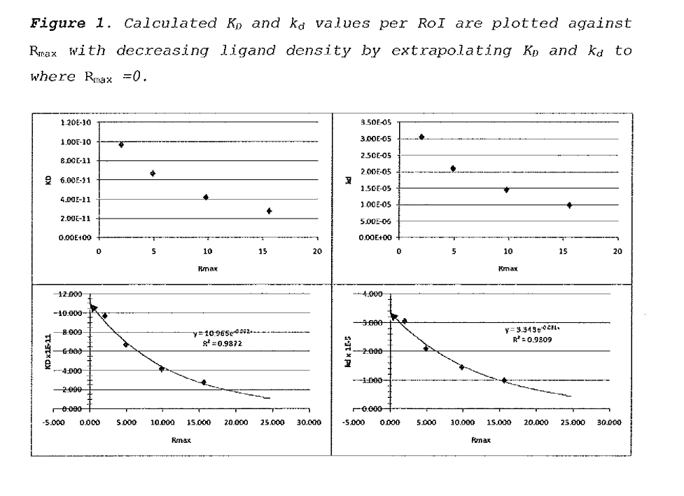

FIG. 1 is a graph wherein the calculated K_{D} and k_{d} values per RoI are plotted against Rₘₐₓ with decreasing ligand density by extrapolating K_{D} and k_{d} to where Rₘₐₓ=0;

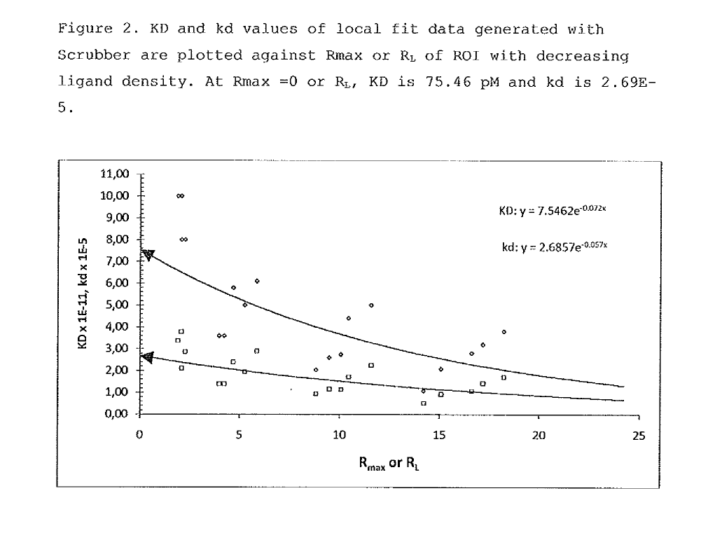

FIG. 2 is a graph wherein K_{D} and k_{d} values of local fit data generated with Scrubber are plotted against Rₘₐₓ or R_{L} of ROI with decreasing ligand density;

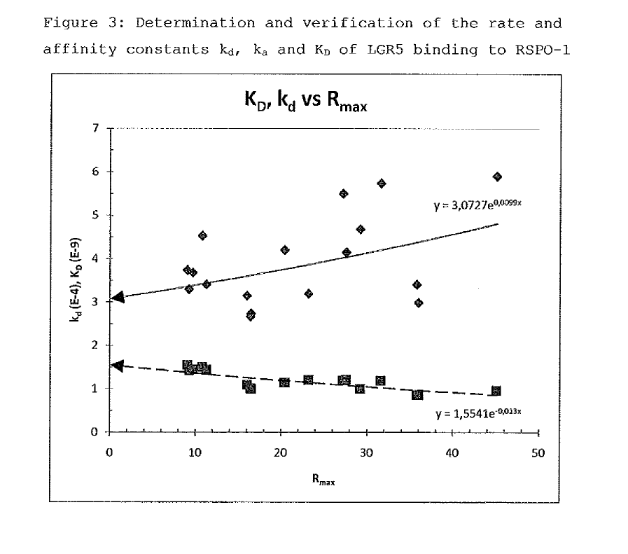

FIG. 3 is a graph for the determination and verification of the rate and affinity constants k_{d}, kₐ and K_{D} of LGR5 binding to RSPO-1; and

FIGS. 4-7 schematically various embodiments of SPR sensor support provides different ligand densities.

### EXAMPLE 1

### Extrapolation of K_{D} and k_{d} at Rmax=0 Using Scrubber Global Curve Fit

Studies were performed for Fab fragments of Herceptin of the full antibodies were analyzed and by applying low concentrations of analyte and weak spots effectively a constant relation of the affinity constant exists between the Fab fragment and full IgG molecule. The biphasic behavior is pronounced especially at high ligand densities, when there is an abundance of epitopes available to the antibody.

Using the method of the invention the K_{D} affinity constant was determined. There is a potential risk for the biphasic effect because of the nature of the IgG structure comprising two binding arms/sites. The risk decreases as the ligand density on an array spot decreases. The fewer epitopes are available, the less likely it becomes that the second “arm” of an IgG molecule can bind after dissociation of the first “arm”. Additionally when few epitopes are available then it will be more likely that a dissociating molecule will diffuse immediately out of the evanescent field of the sensor surface. So the so-called “rebinding effect” is also minimized. Using IBIS MX96 (IBIS By, Enschede, The Netherlands) enables to measure biomolecular interactions to an array of decreasing ligand densities per spot. Per spot the biphasic behavior and rebinding effects will decrease and the effective affinity constant of each spot can be determined using local fit analysis of each interaction curve. In this way the affinity K_{D} can be extrapolated to a theoretical situation where only one ligand molecule is available, thus cancelling the biphasic effect and rebinding effect “in theory” completely. Effectively, the value of K_{D} at a maximal response of 0 is determined.

A CFM array printer of Wasatch Microfluidics, Salt Lake City, UTAH, US was used to create an entire array of spots with the same ligand at serially lower ligand densities. Various ligand densities to 8 spots in the array are generated by exposing the spots to serial diluted (2×) ligand concentrations. Calculated local rate- and affinity parameters for all these ROI by first determining the k_{d} value at the dissociation phase followed by determining the kₐ and Rₘₐₓ value at the association phase can be used to plot calculated k_{d} and K_{D} versus calculated Rₘₐₓ. In FIG. 1, an example using the Ab data set for four ROI is used to extrapolate K_{D} and k_{d} to an Rₘₐₓ of 0. For this extrapolation a conventional exponential fit algorithm is used since at lower ligand densities, more statistical weight has to be given to the data points—these values are theoretically closer to the “true” or intrinsic binding parameters K_{D} and k_{d}.

By entering Rₘₐₓ=x=0 into the fit equation y=ae^{bx}, the exponent of e becomes 0, so the base e becomes 1, leaving only coefficient a as the value of K_{D} and k_{d} at Rₘₐₓ=0. In this case, K_{D} at Rₘₐₓ=0 can be calculated to be 1.10E-10 M, and k_{d} at Rₘₐₓ=0 is calculated to be 3.34E-05, see also FIG. 1. Calculation the binding parameters directly from a single spot may vary from KD=30 pM to 110 pM.

### EXAMPLE 2

### Extrapolation of K_{D} and k_{d} at Rₘₐₓ=0 or R_{L} Using Scrubber Local Curve Fit

The local fit option of Scrubber 2.0 [Biologic Software, Campbell, Australia] can be used to generate a curve fit through more data points, see FIG. 2. Using method described under paragraph 3.4.3 K_{D} and k_{d} at Rₘₐₓ=0 can be calculated from the local fit data generated with Scrubber 2.0. In this case, K_{D} at Rₘₐₓ=0 or R_{L}=0 can be calculated to be 7.55E-11 M, and k_{d} at Rₘₐₓ=0 of R_{L}=0 is calculated to be 2.68E-05.

Table 1 summarizes the affinity parameters obtained for the Herceptin data set using different analysis methods. A good correlation is obtained, with the IBIS local method having the smallest residual values towards the mean.

TABLE 3
Summary of analysis methods used to determine affinity
parameters of the herceptin data set.

| | kₒ × | k_{d} × | K_{D} |
|---|---|---|---|
| Analysis Method | 1e5 | 1e−5 | pM |
| Scrubber, global | 3.24 | 1.89 | 58.30 |
| Scrubber, local | 3.89 | 1.84 | 47.30 |
| IBIS, global | 3.04 | 3.34 | 110.00 |
| IBIS, local | 3.55 | 2.68 | 75.50 |
| Mean | 3.43 | 2.44 | 72.78 |
| SD | 0.37 | 0.71 | 27.40 |
| % (SD:Mean) | 10.86 | 29.30 | 37.64 |
| Mean + SD | 3.80 | 3.15 | 100.17 |
| Mean − SD | 3.06 | 1.72 | 45.38 |

### EXAMPLE 3

### Determination and Verification of the Rate and Affinity Constants k_{d}, kₐ and K_{D} of LGR5 Binding to RSPO-1

The method of the invention is used to determine and verify the rate and affinity constants k_{d}, kₐ and K_{D} of LGR5 binding to RSPO-1. An array of anti-Flag spots anti-Flag antibodies ANTI-FLAG® M2 Monoclonal Antibody from Sigma St. Louis, US with decreasing densities was printed on a preactivated sensor surface (Ssens By, Enschede, the Netherlands) using a continuous flow microspotter (CFM) (Wasatch Microfluidics, Utah, US). Undiluted RSPO-1-FH supernatant cultivated at Hubrecht Laboratory, Utrecht, The Netherlands, was exposed to the array and each anti-Flag spot captured a decreasing density of RSPO-1-FH. After measuring a new baseline for each spot the LGR5-Fc was injected and the sensorgram of each spot was recorded simultaneously. Rₘₐₓ values were correlated to ligand (RSPO-1) densities. However the apparent rate- and affinity constant k_{d}, kₐ and K_{D} will change significantly if rebinding effects, biphasic behavior and/or steric hindrance of the immobilized/captured molecules occurs at the sensor chip surface.

The method of the invention uses all biomolecular interaction data of LGR5 to RSPO-1 responses of the different spots, resulting in a single rate- and affinity constant k_{d}, kₐ and K_{D} at Rₘₐₓ=0 by exponential fitting the apparent k_{d} and K_{D} values to Rₘₐₓ=0. Theoretically this means that the affinity constant of a single ligand molecule to its single analyte molecule is determined in the limit to zero response.

In FIG. 3 the result is shown of the apparent or intrinsic k_{d} and K_{D} values determined from binding curves of serial injections of LGR5 to the array of RSPO-1 captured spots as measured in the IBIS MX96 instrument of IBIS Technologies (Enschede, The Netherlands) using a 1:1 binding model. In SPRint software (IBIS Technologies, Enschede, the Netherlands, version 2) the fitting routines were carried out to fit the apparent k_{d} and K_{D} to Kₘₐₓ=0. By entering Rₘₐₓ=x=0 into the fit equation y=a*e^{bx}, the values of K_{D} and k_{d} at Rₘₐₓ=0 could be determined accurately. The coefficient of the exponential a (y-intercept) resulted in k_{d}=1.55*10⁻⁴ s⁻¹, K_{D}=3.07*10⁻⁹ M respectively (kₐ=5.04*10⁴ M⁻¹s⁻¹) for binding of the LGR5 to RSPO-1.

FIG. 4 shows a sensor support (1) of a micro chip, comprising three surface areas (2), (3), and (4) each comprising a ligand, such as a biomarker for heart disease like galectin-3 or thrombospondin-2, for autoimmune diseases such as rheumatic arthritis, and the like. The areas have different ligand surface densities, such as between 50 RU to 500 RU. Obviously, this sensor support (1) may further comprise surface areas (not shown) with additional ligand densities and for blank, calibration, and a-specific binding.

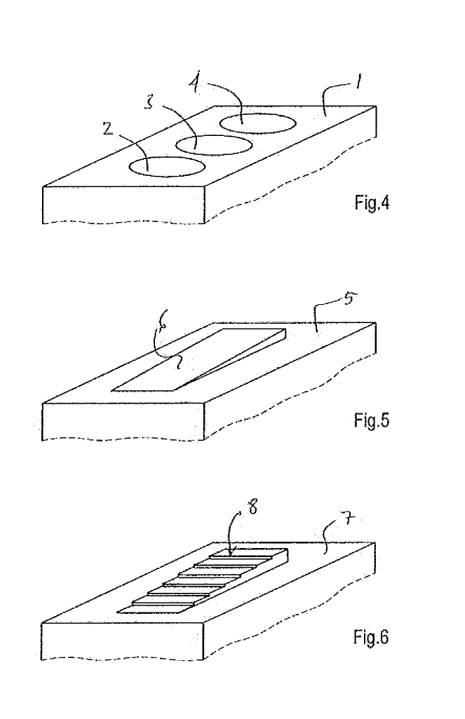

FIG. 5 shows a sensor support (5) comprising an elongated surface area (6) comprising from one (left) side to the other (right) side a gradually increasing ligand surface density or concentration (schematically illustrated by a wedge form). The ligand surface density various from zero to 500 RU over a surface area length of about 1-10 mm. This range may vary for the type of instrument that is used for detection Accordingly, it is possible to monitor simultaneously the binding characteristic over a continuous surface density range and compensate for blank, a-specific binding and optionally for calibration. Such continuous surface density range may be formed by several methods, including but not limited to 3D printing, procedures that use gradual ligand concentration contact times and/or (relative) ligand concentrations, and the like.

FIG. 6 shows a sensor support (7) comprising a surface area (8) comprising a stepwise change in ligand surface density, ranging from 20 to 1000 RU.

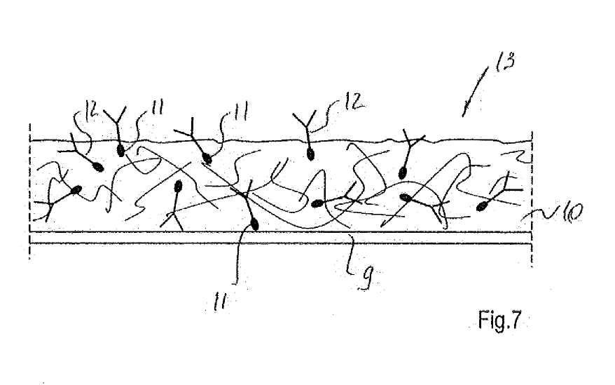

FIG. 7 shows a sensor support (9) such as a gold coating. Attached to the gold coating (9) is surface layer (10) formed by a gel like coating. The gel like coating may include water soluble polymers such as polyethylene oxide and modifications thereof, polyvinyl alcohol, polysugars/polysaccharides, such as dextrans and modifications thereof, heparin, alginate, and the like. The gel like coating (10) has a thickness of about 200-400 nm and forms a 3-dimensional layer. Attached to the gel like coating is an anti-ligand (11), such as streptavidine or protein A. Captured by the anti-ligand (11) is a ligand (12), such as a biotinylated protein or IgG antibody. With the sensor support (9) is created a surface area (13) with substantially the same ligand surface density as the sensor supports of FIG. 4, but with a higher binding capacity. Evidently, such gel like coating may also be provided with the continuous and step wise ligand surface density as shown in FIGS. 5 and 6, respectively, and the ligand may also be immobilized to the gel like coating directly, without the use of an anti-ligand. The anti-ligand can be immobilized in a gradient or the anti-ligand can be equally immobilized and the ligand can be captured in a gradient.

1-15. (canceled)

16. Method for determining intrinsic binding parameters, such as K_{D}, kd and kₐ, of an analyte to a ligand, such as a drug and a protein, a drug and a receptor, and an antibody and antigen, wherein the maximal binding response R_{L} or Rₘₐₓ and at least one binding parameter is determined at at least two different ligand surface densities present on a sensor support, and extrapolating the value of the binding parameter to ligand density=0, characterized by R_{L}=O or Rₘₐₓ=O.

17. Method as claimed in claim 16, wherein the binding parameter is determined at a serial of different ligand surface densities of the ligand.

18. Method as claimed in claim 16, wherein the ligand is distributed in a surface layer permeable to the analyte.

19. Method as claimed in claim 17, wherein the ligand is distributed in a surface layer permeable to the analyte.

20. Method as claimed in claim 16, wherein an anti-ligand of the ligand is coupled to the sensor support and the ligand is captured by the anti-ligand.

21. Method as claimed in claim 19, wherein an anti-ligand of the ligand is coupled to the sensor support and the ligand is captured by the anti-ligand.

22. Method as claimed in claim 16, wherein the analyte is exposed to stepwise or gradual increases of ligand surface densities on the sensor support at a single surface area and/or at distinct surface areas.

23. Method as claimed in claim 21, wherein the analyte is exposed to stepwise or gradual increases of ligand surface densities on the sensor support at a single surface area and/or at distinct surface areas.

24. Method as claimed in claim 16, wherein an analyte concentration is exposed to the different ligand surface densities the same time, and wherein sensorgrams are generated for each ligand concentration.

25. Method as claimed in claim 23, wherein an analyte concentration is exposed to the different ligand surface densities the same time, and wherein sensorgrams are generated for each ligand concentration.

26. Method as claimed in claim 16, wherein at least two ligands are used and at least two analytes are used in a mixture or used sequentially.

27. Method as claimed in claim 25, wherein at least two ligands are used and at least two analytes are used in a mixture or used sequentially.

28. Method as claimed in claim 16, wherein the extrapolation is done by exponential fitting, by local or global fitting, preferably by local fitting.

29. Method as claimed in claim 27, wherein the extrapolation is done by exponential fitting, by local or global fitting, preferably by local fitting.

30. Method as claimed in claim 16, wherein the binding response is measured by surface plasmon resonance.

31. Method as claimed in claim 29, wherein the binding response is measured by surface plasmon resonance.

32. Method for selecting an analyte from a group of analytes for binding to a ligand or group of ligands, comprising the step of:
i) determining an intrinsic binding parameter of the analyte or group of analytes to the ligand or the group of ligands according to a method as claimed by claim 16; and
ii) select the analyte with the better intrinsic binding parameter.

33. Method as claimed in claim 32, wherein the ligand and/or analyte is a pharmacologically active drug, preferably the ligand and/or analyte is related to a physiological status, pathological status, or illness.

34. Ligand and/or analyte selected by the method according to claim 32, for use in medicine, such as for treatment of the physiological status, pathological status or illness.

35. Ligand and/or analyte selected by the method according to claim 33, for use in medicine, such as for treatment of the physiological status, pathological status or illness.

36. Sensor, preferably for surface plasmon resonance sensor, comprising a sensor support to which is attached at least one ligand, optionally via an anti-ligand, comprising a serial of different ligand surface densities of the ligand preferably having a stepwise or gradual increase of ligand surface densities, on the sensor support at a single surface area and/or at distinct surface areas.

37. Sensor as claimed in claim 36, wherein the ligand is distributed in a surface layer permeable to the analyte.

38. Sensor according to claim 36, wherein at least two ligands are attached directly or indirectly to the sensor support or sensor surface layer.

39. Sensor according to claim 37, wherein at least two ligands are attached directly or indirectly to the sensor support or sensor surface layer.
